(19) United States
(12) Patent Application Publication
(10) Pub. No.: US 2009/0214665 A1
(43) Pub. Date: Aug. 27, 2009

(54) Controlled Release Muscarinic Receptor Antagonist Formulation

(76) Inventors: Felix S. Lai, Hayward, CA (US); Richard Ting, San Ramon, CA (US); Lee Foong Siew, San Ramon, CA (US); King Yu-Kuang Chuang, Los Altos, CA (US); Sibel Ucpinar, Cupertino, CA (US)

Correspondence Address: Martin Endres, Esq.;HEDMAN & COSTIGAN, P.C., 1185 Avenue of the Americas, New York, NY, 10036-2601, US

(21) Appl. No.: 12/037,124

(22) Filed: Feb. 26, 2008

(51) Int. Cl. A61K 9/14; A61K 31/137; A61P 21/00

(52) U.S. Cl. 424/497; 424/490; 514/648

(57) ABSTRACT

An oral controlled release pharmaceutical composition for muscarinic receptor antagonist, preferably tolterodine, that employs a drug core, a rapidly disintegrating or rapidly dissolving coating applied to the drug core and a controlled release coating.

### TECHNICAL FIELD

The present invention relates to a stable controlled release formulation of a muscarinic receptor antagonist. More specifically, the present invention relates to a multiparticulate controlled release oral pharmaceutical formulation that contains a muscarinic receptor antagonist, preferably tolterodine, prodrugs, isomers, metabolites or pharmaceutically acceptable salts thereof. The present invention is useful in the treatment of over active bladder with symptoms of urinary urge incontinence, urgency and frequency.

### BACKGROUND OF THE INVENTION

Over active bladder is a disorder that affects millions of individuals and tends to increase with age. It is believed that a main cause of over active bladder arises from the abnormal or uncontrolled activity of the detrusor muscle. This uncontrolled activity is mediated by acetylcholine-induced stimulation of muscarinic receptors. Muscarinic receptor antagonists or antimuscarinics have become a primary class of drug for the treatment of over active bladder. One of the more well known muscarinic receptor antagonists is oxybutynin. Oxybutynin is available as the hydrochloride salt under the tradename DITROPAN® from Alza Corporation.

Although effective in treatment of over active bladder, oxybutynin administration results in a number of adverse side effects such as dry mouth palpitations and constipation. Tolterodine is a newer muscarinic receptor antagonist that is gaining popularity in the treatment of overactive bladder because it exhibits less adverse side effects than oxybutynin.

Tolterodine is commercially available as the tartrate salt under the tradename DETROL® and DETROL® LA. The synthesis of tolterodine is described in a number of publications including U.S. Pat. Nos. 5,382,600; 5,559,269; 5,686,464 and 5,922,914 as well as United States Published Patent Applications 2006/0194876 and 2006/0194987 all of which are incorporated herein by reference. A number of pharmaceutically acceptable salts of tolterodine have also been described in the literature. For example U.S. Pat. No. 7,005,449 discloses a number of tolterodine salts such as tartrate, benzoate, caprate, fumarate, heptanoate, laurate, maleate, pamoate, hydrochloride, mesylate and edisylate, and United States Published Patent Application No. 2006/0194987 discloses the hydrobromide salt.

United States Published Patent Application Nos. 2006/0194987 and 2006/0194987 and EP 325571 disclose methods for preparing the various isomers of tolterodine. These patent publications are incorporated herein by reference.

U.S. Pat. No. 7,005,449 also describes the known major active metabolite of tolterodine, the 5-hydroxymethyl derivative of tolterodine and pharmaceutically acceptable salts of this metabolite.

A number of various dosage forms for muscarinic receptor antagonists have been described in the literature and are commercially available. For example, transdermal patches, oral syrups, immediate release tablets and controlled release osmotic tablets that deliver oxybutynin to a patient are currently being marketed in the United States. According to the United States Food and Drug Administration's publication, Approved Drug Products with Therapeutic Equivalence Evaluations, also known as the Orange Book, oxybutynin is currently available in an oral extended release form known as DITROPAN® XL which is believed to be an osmotic tablet as described in U.S. Pat. No. 5,840,754.

A number of pharmaceutical dosage forms for delivery of tolterodine to a patient are also described in the literature such as transdermal patches, inhalation formulations (U.S. Pat. No. 7,005,449) and liquid formulations (U.S. Pat. No. 7,101,888). U.S. Pat. No. 7,101,888 reports that tolterodine exhibits chemical instability in aqueous media at neutral to alkaline pH levels.

Immediate release tablets and extended release capsules of tolterodine are currently available in the United States under the tradename DETROL® and DETROL® LA. The Orange Book currently lists U.S. Pat. Nos. 6,770,295; 6,630,162 and 6,911,217 and other patents for the DETROL® LA product. The dosage form described in U.S. Pat. No. 6,911,217 is a multiparticulate dosage form that consists of an inert core coated with a water insoluble polymer. The water insoluble polymer layer is then coated with a drug layer that contains tolterodine tartrate. The drug layer is then directly coated with a controlled release coating.

Other multiparticulate dosage forms of tolterodine have also been described in the literature. For example, PCT application WO 2004/105,735 describes a tolterodine pellet that consists of an inert core coated with a drug layer containing tolterodine and overcoated with a controlled release coating. United States Published Patent Application No. 2007/0248670 describes a formulation similar to that described in U.S. Pat. No. 6,911,217 except a water soluble polymer is used to coat the inert core prior to the application of the drug layer and the controlled release coating.

All known prior art controlled release formulations of tolterodine employ a very complicated multistage manufacturing process and potentially expose the tolterodine to adverse conditions. For example, these prior art processes expose the tolterodine to potential interactions and/or degradation with the materials used in the controlled release coatings. These prior art processes also require a drug layering step that often requires the dissolving or suspending of the tolterodine in a solvent and spraying the solution or suspension onto an inert carrier. The conditions used in the drug layering step further expose the tolterodine to conditions that potentially could be adverse to the drug.

It is an object of the present invention to provide a stable oral controlled release dosage form for muscarinic receptor antagonists that avoids the potential adverse interactions between the muscarinic receptor antagonist and the controlled release coatings.

It is a further object of the present invention to provide a stable oral controlled release dosage form for muscarinic receptor antagonists that avoids, i.e. does not employ, a step of layering the drug onto an inert substrate.

It is still a further object of the present invention to provide a stable oral controlled release dosage form that employs a novel coating system to control release of the drug from the dosage form.

It is another object of the present invention to provide a stable oral controlled release dosage form that exhibits similar in vivo characteristics, i.e. no food effect, when administered with and without food.

Finally, it is an object of the present invention to provide a controlled release dosage form that exhibits an improved bioavailability when compared to conventional controlled release muscarinic receptor antagonists dosage forms such as DETROL®LA.

These and other objects of the present invention will become apparent from a review of the appended specification.

### SUMMARY OF THE INVENTION

The present invention accomplishes the above objects and others by providing a novel dosage form for muscarinic receptor antagonists that comprises a drug core, a rapidly disintegrating or rapidly dissolving coating surrounding the core and a controlled release coating surrounding the rapidly disintegrating or rapidly dissolving coating.

The drug core may be prepared by layering the muscarinic receptor antagonists onto an inert carrier, however, in a preferred embodiment the muscarinic receptor antagonists are mixed with pharmaceutically acceptable excipients and formed into a core by conventional methods such as compression or extrusion/spheronization.

The rapidly disintegrating or rapidly dissolving coating preferably comprises a low molecular weight water soluble polymer that will surround the drug core. The rapidly disintegrating or rapidly dissolving coating may protect the muscarinic receptor antangonist from potential adverse interactions with the controlled release coating materials (solids and solvents) during the application of the controlled release coating and during storage. The rapidly disintegrating or rapidly dissolving coating may also protect the muscarinic receptor antagonists in the core from moisture during storage. The rapidly disintegrating or rapidly dissolving coating may also provide a surface, preferably a smooth and uniform surface, which aids in the application and adhesion of the subsequent controlled release coating.

The rapidly disintegrating or rapidly dissolving coating does not contain any muscarinic receptor antagonist or other pharmaceutically active drug and is a separate and distinct layer between the drug core and the controlled release coating of the present invention. The rapidly disintegrating or rapidly dissolving coating should disintegrate or dissolve within 30 minutes, preferably in less than 20 minutes and most preferably in less than 15 minutes when it is exposed to an aqueous fluid environment.

The controlled release coating is applied to the rapidly disintegrating or rapidly dissolving coating. The controlled release coating comprises a water insoluble film forming polymer and a pore forming agent. In a preferred embodiment, the controlled release coating employs a pH dependent material, preferably an enteric material or polymer that dissolves at pH level above 4.5. The pH dependent material may be formulated into the controlled release coating with the water insoluble film forming polymer and be a pore forming agent however, in a preferred embodiment the pH dependent material is applied as a separate and distinct coating over the controlled release coating.

In one embodiment, the dosage form of the present invention exhibits similar pharmacokinetic values when administered to a patient under fed and fasting conditions. Examples of the similar pharmacokinetic values are the area under the plasma concentration time curve (AUC); the maximum plasma concentration (Cₘₐₓ) and time to maximum plasma concentration (Tₘₐₓ).

In an alternate embodiment of the present invention, the bioavailability of the controlled release dosage form is increased compared to a conventional extended release muscarinic receptor antagonist. More specifically, in this alternative embodiment, the 90% confidence interval of the AUC and Cₘₐₓ is greater than 125 compared to the AUC and Cₘₐₓ of a conventional extended release muscarinic receptor antagonist dosage form.

### BRIEF DESCRIPTION OF THE DRAWINGS:

FIG. 1 is a graph of the in vitro dissolution profile of a dosage form of Examples 1 and 2 in 800 ml of simulated gastric fluid (SGF) using a United States Pharmacopoeia (USP) Apparatus I (basket) at 100 rpms.

FIG. 2 is a graph of the in vitro dissolution profile of a dosage form of Examples 1 and 2 in 800 ml of an aqueous pH 6.8 buffered media using a USP Apparatus I (basket) at 100 rpms.

FIG. 3 is the mean plasma concentration profile for Example 1 from a single dose study conducted under fasting conditions with n=66

FIG. 4 is the mean plasma concentration profile for Example 1 from a single dose study conducted under fed conditions with n=13

FIG. 5 is the mean plasma concentration profile for Example 2 from a single dose study conducted under fasting conditions with n=12

FIG. 6 is the mean plasma concentration profile for Example 2 from a single dose study conducted under fed conditions with n=11

### DETAILED DESCRIPTION OF THE INVENTION:

The subject invention concerns a stable controlled release oral pharmaceutical formulation or dosage form comprising a muscarinic receptor antagonist such as benztropine, darfenacin, oxybutynin, solifenacin, tolterodine trihexypheidyl or triotropium. The preferred muscarinic receptor antagonists are oxybutynin and tolterodine, with the more preferred being tolterodine, its isomers, and pharmaceutically acceptable salts therefore or its active metabolite, the 5-hydroxymethyl derivative.

The most preferred muscarinic receptor antagonist for use in the present invention is tolterodine tartrate and more specifically (R)-N,N-diisopropyl-3-(2-hydroxy-5 methylphenyl)-3-phenylpropanamine L-hydrogen tartrate. Unless otherwise indicated, the term “tolterodine” as used throughout this specification includes racemic mixtures, as well as the individual isomers, pharmaceutically acceptable salts and the active metabolite.

The muscarinic receptor antagonist is formulated into a solid oral dosage form in accordance with the present invention by mixing the muscarinic receptor antagonist with conventional pharmaceutical excipients to prepare a core. The core is then coated with a rapidly disintegrating or rapidly dissolving coating. This rapidly disintegrating or rapidly dissolving coating may protect the muscarinic receptor antagonist in the core from the subsequently applied controlled release coating and may also protect the drug core from moisture during application of the controlled release coating and during storage. Once the rapidly disintegrating or rapidly dissolving coating is applied to the dosage form, a controlled release coating is applied to the dosage form. The controlled release coating will delay and control the release of the muscarinic receptor antagonist drug from the core of the dosage form over time.

The final dosage form of the present invention can be a tablet or capsule. A preferred embodiment of the present invention is a multiparticulate tablet or capsule that comprises a plurality of controlled release particles in accordance with the present invention. As used herein, the term particle includes small individual units that comprise at least the drug and one pharmaceutically acceptable excipient. The particles may range in size from about 0.1 mm to about 3.0 mm, preferably about 0.5 to about 1.5 mm. The particles may be coated granules, mini tablets, beads or pellets.

In the case of granules, the granule may be formed by conventional wet or dry granulation techniques wherein the muscarinic receptor antagonist is mixed with at least one pharmaceutically acceptable excipient such as a filler to create a granule that will be coated as described in detail below. The granule may be irregularly shaped or approximately spherical.

In the case of mini tablets, the muscarinic receptor antagonist is mixed with conventional pharmaceutical excipients and compressed into small tablets using conventional techniques such as a rotary tablet press which can then be coated as described below.

The particles may also be beads or pellets prepared by layering the muscarinic receptor antagonist onto an inert carrier such as a sugar seed, a microcrystalline cellulose seed or a glass bead. The drug layered beads or pellets may also be coated as described below.

In a preferred embodiment, the particles are prepared by extrusion spheronization wherein the muscarinic receptor antagonist and at least one pharmaceutically acceptable excipient are mixed and/or granulated. The resulting mixture is then extruded into small discrete units, spheronized and sized to acceptable size ranges. The spheronized cores are then coated as described below.

The acceptable pharmaceutical excipients employed in the core can be any of the well known excipients describe in the Handbook of Pharmaceutical Excipients or the United States Pharmacopeia. Some of the preferred excipients are binders, fillers, lubricants and glidants.

Acceptable binders are, for example, celluloses such as hydroxypropyl methycellulose, hydroxypropyl cellulose and carboxymethycellulose sodium;

polyvinylpyrrolidone; sugars; starches and other pharmaceutically acceptable substances with cohesive properties.

Acceptable fillers, sometimes referred to as diluents, include sugars such as lactose, dextrose, sucrose, maltose, or microcrystalline cellulose and the like.

Examples of lubricants and glidants that may be used in the present invention include talc, magnesium stearate, calcium stearate, stearic acid, hydrogenated vegetable oils, polyethylene glycols, silicon dioxide and mixtures of the foregoing.

In one embodiment of the present invention, the core is prepared without the use of film forming polymers and comprises the muscarinic receptor antagonist and non-film forming materials such as sugars, preferably lactose and/or microcrystalline cellulose.

Once the muscarinic receptor antagonist core is prepared, it is coated with a rapidly disintegrating or rapidly dissolving coating. The rapidly disintegrating or rapidly dissolving coating is applied to the cores by conventional procedures such as the use of a coating pan or fluidized bed apparatus using water and/or conventional organic solvents for the coating solution. The materials for the rapidly disintegrating or rapidly dissolving coating are chosen from among pharmaceutically acceptable water soluble or water dispersible inert compounds or polymers used for film coating applications such as sugar, polyethylene glycol, povidone, polyvinyl alcohol, hydroxypropyl cellulose, methylcellulose, hydroxymethyl cellulose, hydroxypropyl methylcellulose, polyvinyl acetal diethylaminoacetate and the like. An especially preferred material for the rapidly disintegrating or rapidly dissolving coating is commercially available from Colorcon under the tradename OPADRY AMB. It is believed that OPADRY AMB comprises polyvinyl alcohol, talc, lecithin soya and xanthan gum.

In one embodiment of the present invention, the rapidly disintegrating or rapidly dissolving coating should be a film that exhibits a low water vapor transmission rate (WVTR). The WVTR can be measured using a WPA Water Permeability Analyzer commercially available from VTI Corporation. The WVTR is determined by placing a cast film in a 6.39 square centimeter cell at a nitrogen flow rate of 200 cubic centimeters/minute. The films as measured at 25° C./60% relative humidity (RH), 25° C./80% RH and 40° C./75% RH. The resistance of the film to water vapor permeation is calculated from slope of the WVTR versus time plot. The preferred rapidly disintegrating or rapidly dissolving coating should exhibit a slope that is less than 60, preferably less than 40 and most preferably less than 20 under all three test conditions.

After the rapidly disintegrating or rapidly dissolving coat is applied to the core, a controlled release coating is applied to the rapidly dissolving or rapidly dissolving coating.

Materials that are useful in forming the controlled release coating are water insoluble film forming polymers such as ethylcellulose, polymethacrylates such as ethyl acrylate/methyl methacrylate copolymer and amino methacrylate copolymers, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate and cellulose acetate butyrate. Other suitable polymers are described in U.S. Pat. Nos. 4,780,318; 5,330,766; and 6,911,217 which are incorporated herein by reference.

The controlled release coating should also comprise a pore forming agent. Examples of possible pore forming agents are sodium chloride, potassium chloride, sucrose, sorbitol, mannitol, polyethylene glycols (PEG), propylene glycol, hydroxypropyl cellulose, hydroxypropyl methycellulose, polyvinyl alcohols, methacrylic acid copolymers, poloxamers (such as LUTROL F68, LUTROL F127 and LUTROL F108 which are commercially available from BASF) and mixtures thereof. It is also possible to employ pH dependent materials as the pore forming agents. The pH dependent materials are often known as enteric materials and do not dissolve in acidic environments. Generally, the enteric materials do not dissolve until they encounter an aqueous media with a pH of about 4.5 or higher, preferably about 5.0 or higher, and most preferably about 5.5 and higher. Representative examples of pH dependent materials that can be used include cellulose acetate phthalate, hydroxypropylmethyl cellulose phthalate, polyvinyl acetate phthalate, carboxymethylethylcellulose, Eudragit L (poly(methacrylic acid, methylmethacrylate), 1:1 ratio, MW (No. Av. 135,000—USP Type A) or Eudragit S (poly(methacrylic acid, methylmethacrylate, 1:2 ratio, MW (No. Av. 135,000—USP Type B) and mixtures thereof.

The controlled release coating may also include conventional processing aids such as lubricants and glidants described above as well as plasticizers. Suitable plasticizers include acetyl triethyl citrate, dibutyl phthalate, tributyl citrate, triethyl citrate, acetyl tributyl citrate, propylene glycol, triacetin, polyethylene glycol and diethyl phthalate.

In a preferred embodiment, the controlled release coating comprises a first controlled release coating that comprises the water insoluble polymer and a pore forming agent and a second controlled release coating, preferably a delayed release coating, that comprises a pH dependent material or polymer. In this embodiment, it is preferred that the pore forming agent in the first controlled release coating be a water soluble polymer.

In the case of a multiparticulate dosage from, after the controlled release coating is applied, a plurality of the controlled release particles can be filled into a hard or soft gelatin capsule or mixed with conventional tableting excipients and compressed into a tablet.

The final dosage form of the present invention may also include an immediate release amount of the muscarinic receptor antagonist if desired. The immediate release component can be in the form of free drug added to the capsule or tablet, particles of the muscarinic receptor antagonist as described above that have not been coated with the controlled release coating or an immediate release layer of the muscarinic receptor antagonist applied to the controlled release coating of the tablets or particles.

An embodiment of a dosage form in accordance with the present invention will have the following composition based upon the total weight of the final dosage form:

Core:

| | | | |
|---|---|---|---|
| drug | 0.5-15% | (1-10% | preferred) |
| excipient | 40-90% | (50-85% | preferred) |
| Rapidly Disintegrating or | 1-15% | (2-12% | preferred) |
| Rapidly Dissolving Coating: | | | |

Controlled Release Coating:

| | | | |
|---|---|---|---|
| insoluble polymer | 0.5-10% | (1-5% | preferred) |
| pore forming agent | 0.5-20% | (1-15% | preferred) |
| plasticizer | 0-10% | (0.5-5% | preferred) |
| lubricant/glidant | 0-15% | (0.5-10% | preferred) |

Another preferred embodiment of the dosage form in accordance with the present invention will have the following composition based upon the total weight of the final dosage form:

Core:

| | | | |
|---|---|---|---|
| drug | 0.5-15% | (1-10% | preferred) |
| excipient | 40-90% | (50-85% | preferred) |
| Rapidly Disintegrating or | 1-15% | (2-12% | preferred) |
| Rapidly Dissolving Coating: | | | |

First Controlled Release Coating:

| | | | |
|---|---|---|---|
| insoluble polymer | 0.5-10% | (1-5% | preferred) |
| pore forming agent | 0.25-10% | (0.5-8% | preferred) |
| plasticizer | 0-5% | (0.25-3% | preferred) |
| lubricant/glidant | 0-5% | (0.25-3% | preferred) |

Second Controlled Release Coating:

| | | | |
|---|---|---|---|
| pH dependent polymer | 1-20% | (2.5-15% | preferred) |
| plasticizer | 0-5% | (0.25-3% | preferred) |
| lubricant/glidant | 0-10% | (0.5-10% | preferred) |

The dosage form of the present invention should exhibit the following in vitro dissolution profile when tested in a United States Pharmacopeia (USP) type 1 apparatus (basket) at 100 rpms in 800 ml of an aqueous pH 6.8 phosphate buffer and at 37° C.:

| Time (hours) | Preferred | Most Preferred |
|---|---|---|
| 2 | 0-40% | 5-30% |
| 4 | 20-80% | 30-75% |
| 8 | NLT-60% | NLT-70% |

NLT = NOT LESS THAN

In an especially preferred embodiment of the present invention, the dosage form will release 0-20% of the tolterodine within 2 hours, 20-50% of the tolterodine within 4 hours and 50-75% of the tolterodine within 6 hours of testing in a pH 6.8 aqueous media as described above.

The dosage form of the present invention should also exhibit the following in vitro dissolution profile when tested in a USP type 1 apparatus at 100 rpms in 800 ml of simulated gastric fluid (SGF) and at 37° C.:

| Time (hours) | Preferred | Most Preferred |
|---|---|---|
| 2 | 0-40% | 0-30% |
| 4 | 1-50% | 5-45% |
| 8 | NLT-20% | NLT-30% |

NLT = NOT LESS THAN

In an especially preferred embodiment of the present invention, the dosage form will release 0-15% of the tolterodine within 2 hours, 5-20% of the tolterodine within 4 hours and 10-40% of the tolterodine within 6 hours of testing in SGF as described above.

Certain embodiments of the present invention were tested in vivo according to the United States Food and Drug Administrations (FDA) procedures for measuring bioavailability and bioequivalence of an orally administered drug. A general description of the in vivo testing procedures can be found in the FDA documents entitled “Guidance for Industry—Bioavailability and Bioequivalence Studies for Orally Administered Drug Products-General Considerations” March 2003 and/or “Guidance for Industry-Food-Effect Bioavailability and Fed Bioequivalence Studies” December 2002 which are incorporated herein by reference. The data from the in vivo studies was analyzed using standard statistical procedures such as outlined in the FDA documents entitled “Statistical Procedures for Bioequivalence Studies Using a Standard Two-Treatment Crossover Design” July 1992 and/or “Statistical Approaches to Establishing Bioequivalence” which are incorporated herein by reference.

The in vivo testing of one embodiment of the present invention demonstrated that the present invention can exhibit a similar bioavailability when tested under fed conditions and fasting conditions. More specifically, the in vivo testing demonstrated that an embodiment of the present invention exhibits similar area under the mean plasma concentration time curves (AUC), similar mean peak plasma concentrations (Cₘₐₓ) and similar time to maximum plasma concentrations (Tₘₐₓ) when the dosage form is administered to patients in the morning under fed and fasting conditions. Stated another way, the in vivo testing has confirmed that an embodiment of the present invention is bioequivalent to itself under fed and fasting conditions (that the 90% confidence interval of the Cₘₐₓ and AUC values is within the range of 80 to 125). This lack of food effect exhibited by the present invention is significant because it allows the dosage form to be administered to patients at any convenient time without the fear that the drug may be released too slowly or too quickly.

The in vivo testing has also demonstrated that an alternative embodiment of the present invention exhibits an increased bioavailability compared to conventional controlled release tolterodine dosage forms such as DETROL® LA. For example, tolterodine dosage forms prepared in accordance with certain embodiments of the present invention exhibit a Cₘₐₓ and an AUC, when compared to DETROL® LA, wherein the 90% confidence interval for the Cₘₐₓ and/or AUC is greater than 125. In addition, a tolterodine dosage form prepared in accordance with certain embodiments of the present invention will exhibit a Cmax that is at least 2500 ng/ml or greater, preferably at least 3000 ng/ml or greater based upon a one a day administration of 4 mg of tolterodine. The dosage form will also exhibit an AUC that is about 35,000 ng*hr/ml or greater, preferably about 40,000 ng*hr/ml or greater based upon a once a day administration of 4 mg of tolterodine. This increased bioavailability is surprising and potentially beneficial because it may allow the administration of smaller doses of tolterodine to a patient without reducing the therapeutic effects.

### DESCRIPTION OF THE PREFERRED EMBODIMENTS:

The following are provided by way of example only and are by no means intended to be limiting.

### EXAMPLE 1

A 4 mg controlled release multiparticulate tolterodine tartrate capsule in accordance with the present invention was prepared as follows:

### Core

Approximately 2.56 kg of tolterodine tartrate, 70.08 kg of microcrystalline cellulose (Avicel PH 101) and 23.36 kg of lactose monohydrate NF were de-lumped using a Comil equipped with a 0.039 inch round screen. The de-lumped materials were then granulated in a high shear granulator with approximately 80 kg of purified USP water. The granulated material was collected and extruded with a 1.0 mm hole screen and spheronized with a 3 mm cross hatch disc.

The spheronized cores were dried in an oven at 60° C. for about 16-20 hours.

The dried spheronized cores were screened through 16 and 24 mesh screens and the dried spheronized cores retained on the 24 mesh screen were collected.

### Rapidly Disintegrating or Rapidly Dissolving Coating

Approximately 80 kg of the dried tolterodine tartrate spheronized cores were coated with approximately 8.0 kg of OPADRY AMB White (80W68912) solids using a fluidized bed coater with a Wurster insert. The OPADRY AMB White was applied by dispersing the 8.0 kg of OPADRY AMB White in approximately 72 kg of purified USP water prior to spraying into the fluidized bed.

Once the OPADRY AMB White/water suspension was consumed, the coated tolterodine cores were dried in the fluidized bed coater for approximately 20-30 minutes.

The dried coated cores were screened through 14 and 24 mesh screens and the coated cores retained on the 24 mesh screen were collected.

### Controlled Release Coating

A controlled release suspension comprising approximately 0.90 kg of ethylcellulose NF (ETHOCEL Standard 20 Premium); 0.30 kg of hypromellose, Type 2910 USP; 0.06 silicon dioxide NF (SYLOID 244 FP); 0.24 kg triethyl citrate, NF and 28.80 kg of ethyl alcohol USP (90 proof) was prepared. The controlled release suspension was applied to approximately 20 kg of the OPADRY AMB coated cores using a fluidized bed coater with a Wurster insert. Once the controlled release coating suspension was consumed, the controlled release coated tolterodine cores were dried at 60° C. for approximately 20 minutes in the fluidized bed coater.

### Delayed Release Coating

A delayed release suspension comprising approximately 2.15 kg of hypromellose phthalate, NF (HPMCP: HP-55) 0.09 kg acetyltributyl citrate, NF); 1.07 kg talc USP (IMP1885L, Talc Imperial USP BC) and 29.79 kg of acetone NF was prepared. The delayed release coating suspension was applied to approximately 21.50 kg of the controlled release coated cores using a fluidized bed coater with a Wurster insert. Once the delayed release coating suspension was consumed, the delayed release coated tolterodine cores were dried at 60° C. for approximately 30 minutes in the fluidized bed coater.

The dried delayed release coated cores were screened through 14 and 24 mesh screens and the delayed release coated cores retained on the 24 mesh screen were collected.

The screened delayed release coated cores were dusted with approximately 0.13 kg of talc and filled into hard gelatin capsules to create a controlled release oral capsule containing approximately 4 mg of tolterodine tartrate. The final dosage form had the following composition:

| Ingredient | % (w/w) | mg/capsule |
|---|---|---|
| Tolterodine Tartrate | 1.94 | 4.0 |
| Microcrystalline Cellulose | 53.23 | 109.50 |
| Lactose Monohydrate | 17.74 | 36.50 |
| OPADRY AMB White | 7.29 | 15.00 |
| Ethylcellulose | 3.61 | 7.42 |
| Hypromellose | 1.21 | 2.48 |
| Silicon Dioxide | 0.24 | 0.50 |
| Triethyl Citrate | 0.96 | 1.98 |
| Hypromellose Phthalate | 8.62 | 17.74 |
| Acetyltributyl Citrate | 0.35 | 0.71 |
| Talc | 4.81 | 9.89 |

### EXAMPLE 2

A 4 mg controlled release multiparticulate tolterodine tartrate capsule was prepared according to the procedure described in Example 1 except the delayed release coating was not applied. The final 4 mg capsule had the following composition:

| Ingredient | % (w/w) | mg/capsule |
|---|---|---|
| Tolterodine Tartrate | 2.3 | 4.0 |
| Microcrystalline Cellulose | 62.3 | 109.50 |
| Lactose Monohydrate | 20.8 | 36.50 |
| OPADRY AMB White | 8.5 | 15.00 |
| Ethylcellulose | 3.5 | 6.19 |
| Hypromellose | 1.2 | 2.06 |
| Triethyl Citrate | 0.9 | 1.65 |
| Talc* | 0.5 | 0.87 |

*all talc was dusted onto the controlled release coated cores

The dosage forms of Examples 1 and 2 exhibited the following in vitro dissolution profile when tested in a United States Pharmacopoeia (USP) type 1 apparatus (basket) at 100 rpms in 800 ml of SGF and at 37° C.:

| | Example 1 | Example 2 | DETROL ® LA |
|---|---|---|---|
| Time | % released | % released | % released |
| 1 | 0.3 | 6.2 | 13.3 |
| 2 | 0.6 | 18.2 | 19.2 |
| 3 | 2.5 | 29.7 | 25.0 |
| 4 | 8.1 | 39.5 | 31.23 |
| 5 | 16.6 | 47.3 | |
| 7 | 31.4 | 58.0 | 50.53 |
| 9 | 40.1 | | |
| 10 | | 67.8 | 66.8 |
| 12 | 49.1 | 72.8 | |

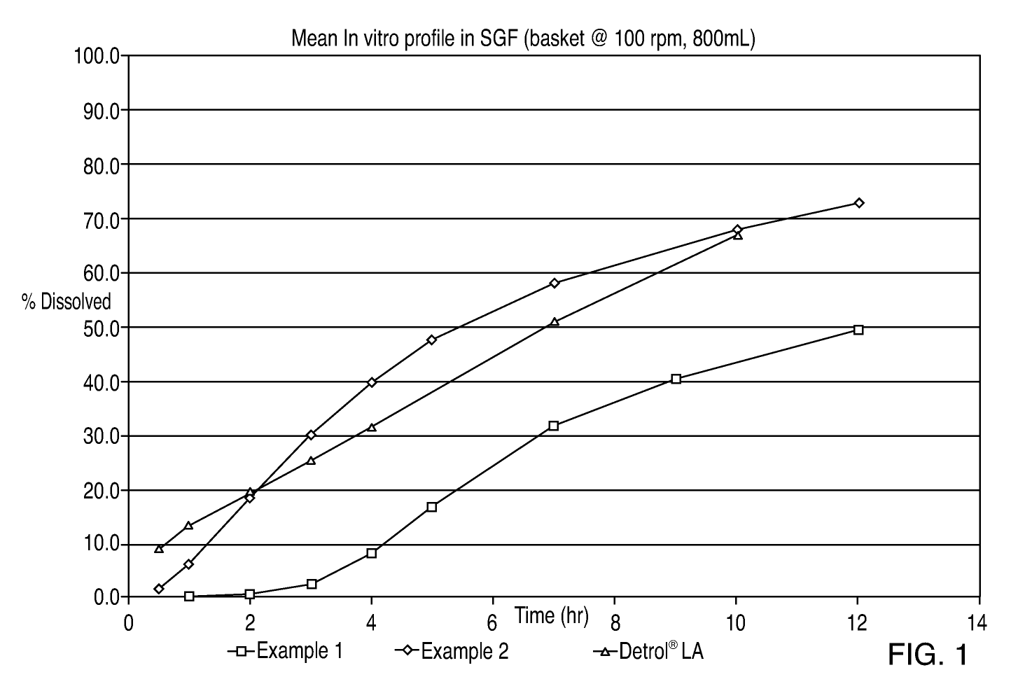

A graph of the above results is shown in FIG. 1. The above results for Example 1 and DETROL®LA were obtained using three (3) dissolution vessels. The above results for Example 2 were obtained using six (6) dissolution vessels.

The dosage forms of Examples 1 and 2 exhibited the following in vitro dissolution profile when tested in a United States Pharmacopeia (USP) type 1 apparatus (basket) at 100 rpms in 800 ml of an aqueous pH 6.8 phosphate buffer and at 37° C.:

| | Example 1 | Example 2 | DERTOL ® LA |
|---|---|---|---|
| Time | % released | % released | % released |
| 1 | 1.0 | 8.0 | 14.0 |
| 2 | 13.9 | 24.9 | 38.0 |
| 3 | 31.5 | 39.9 | 60.0 |
| 4 | 45.8 | 51.9 | 75.0 |
| 5 | 57.2 | 61.1 | |
| 7 | 73.6 | 75.0 | 91.0 |
| 10 | 88.1 | 88.4 | |
| 11 | | | 94.0 |
| 12 | 94.0 | 94.5 | |
| 13 | | | 94.0 |

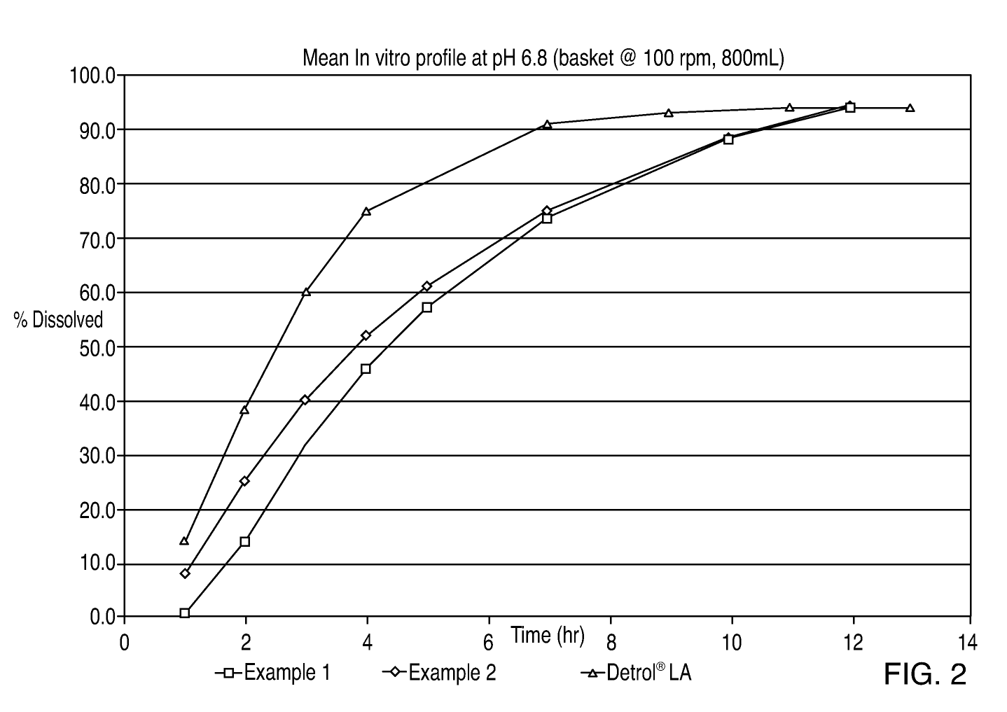

A graph of the above results is shown in FIG. 2. The above results for Example 1 and Example 2 were obtained using three (3) dissolution vessels. The above results for DETROL®LA were obtained using twelve (12) dissolution vessels.

Example 1 and DETROL®LA a commercially available extended release capsule version of tolterodine tartrate were also tested in vivo according to standard FDA bioequivalency testing procedures. The results of the single dose cross over study are as follows:

MEAN PHARMACOKINETIC PARAMETERS (FASTED) (n = 66)

| Parameter | Example 1 | DETROL ® LA |
|---|---|---|
| Tₘₐₓ (hr) | 5.5 | 5.0 |
| Cₘₐₓ (pg/ml) | 1852.35 | 1834.59 |
| AUCₒ₋ₜ (pg * hr/ml) | 30547.89 | 29321.60 |

MEAN PHARMACOKINETIC PARAMETERS (FED) (n = 13)

| Parameter | Example 1 | DETROL ® LA |
|---|---|---|
| Tₘₐₓ (hr) | 7.0 | 5.0 |
| Cₘₐₓ (pg/ml) | 1710.49 | 1696.52 |
| AUCₒ₋ₜ (pg * hr/ml) | 21818.54 | 23930.98 |

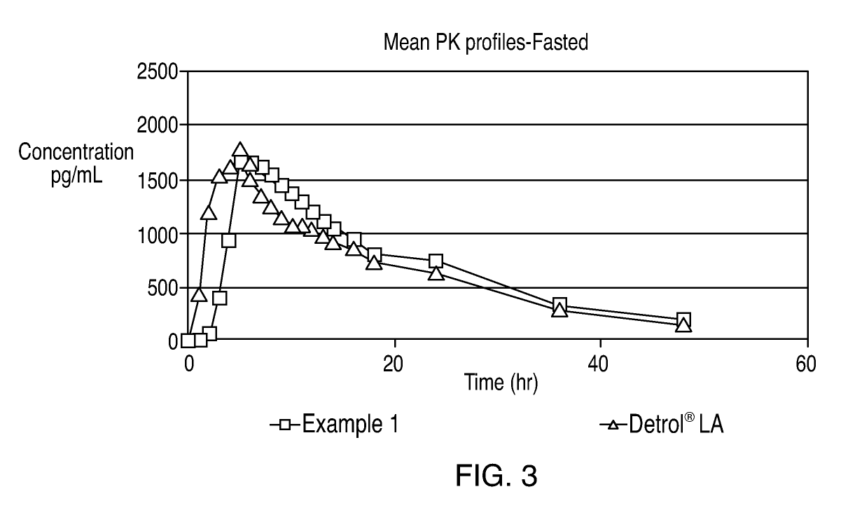

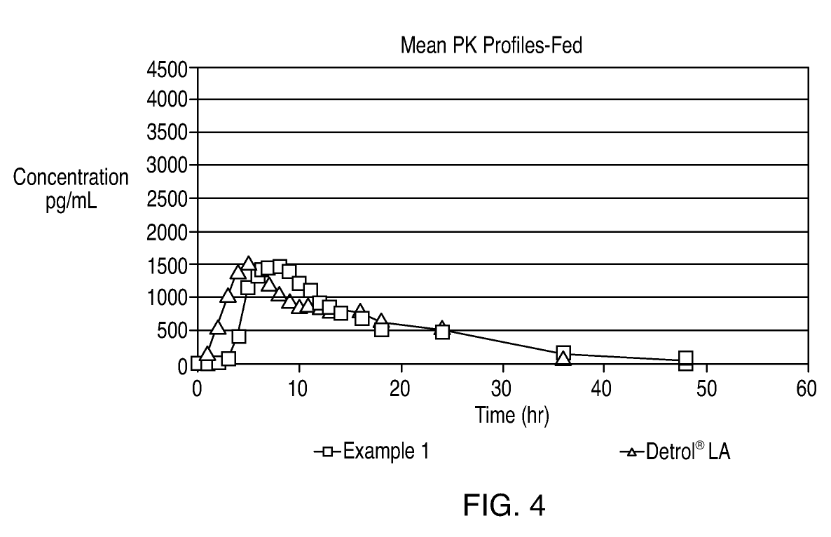

The mean plasma concentration time graph for the above described in vivo testing is shown in FIGS. 3 and 4. The above data clearly shows that the presence of a delayed release coating allows the dosage form of the present invention to exhibit similar bioavailability under fed and fasting conditions.

Example 2 and DETROL®LA a commercially available extended release capsule version of tolterodine tartrate were also tested in vivo according to standard FDA bioequivalency testing procedures. The results of a single dose cross over study are as follows:

MEAN PHARMACOKINETIC PARAMETERS (FASTED) (n = 12)

| Parameter | Example 2 | DETROL ® LA |
|---|---|---|
| Tₘₐₓ (hr) | 5.3 | 5.4 |
| Cₘₐₓ (pg/ml) | 2175.3 | 1769.1 |
| AUCₒ₋ₜ (pg * hr/ml) | 28176.3 | 29028.6 |

MEAN PHARMACOKINETIC PARAMETERS (FED) (n = 11)

| Parameter | Example 1 | DETROL ® LA |
|---|---|---|
| Tₘₐₓ (hr) | 4.9 | 6.60 |
| Cₘₐₓ (pg/ml) | 4525.9 | 1829.6 |
| AUCₒ₋ₜ (pg * hr/ml) | 49682.7 | 30353.54 |

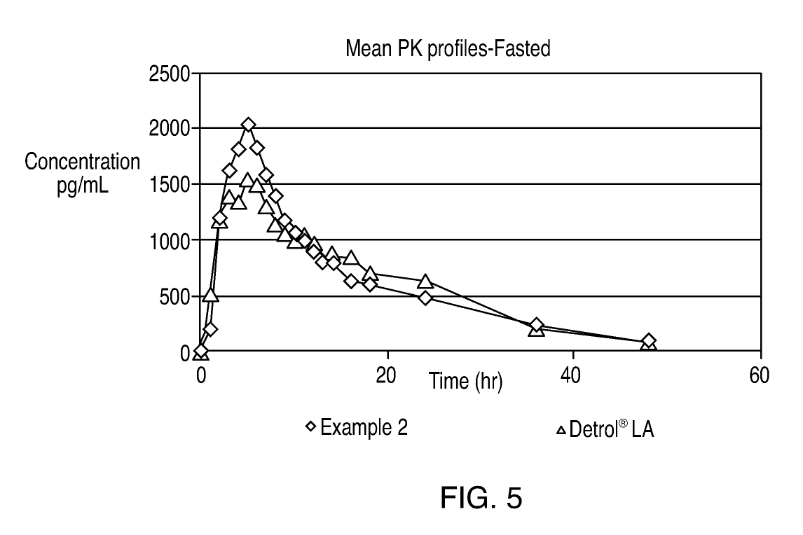

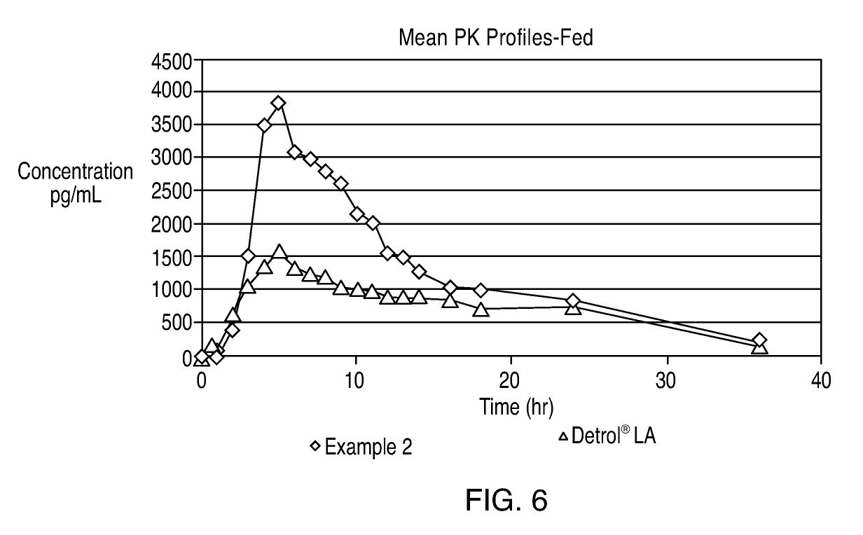

The mean plasma concentration time graph for the above described in vivo testing is shown in FIGS. 5 and 6. The above data on Example 2 shows that the absence of a delayed release coating can result in an extended release dosage form with improved bioavailability, especially when administered with food.

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

1. A stable controlled release oral pharmaceutical composition comprising:
(a) a core comprising a therapeutically effective amount of tolterodine and at least one pharmaceutically acceptable excipient;
(b) a rapidly disintegrating or rapidly dissolving coating applied directly to the core; and
(c) a controlled release coating applied to the rapidly disintegrating or rapidly dissolving coating wherein the controlled release coating comprises a water insoluble film forming polymer and a pore forming agent.

2. The pharmaceutical composition as defined in claim 1 wherein the pharmaceutical composition is a multiparticulate dosage form wherein the dosage form comprises a plurality of particles that comprise elements (a), (b) and (c).

3. The pharmaceutical composition as defined in claim 1 wherein the tolterodine is a pharmaceutically acceptable salt of tolterodine.

4. The pharmaceutical composition as defined in claim 1 wherein the tolterodine is tolterodine tartrate.

5. The pharmaceutical composition as defined in claim 1 wherein the rapidly disintegrating or rapidly dissolving coating exhibits a low water vapor transmission rate as determined by a water permeability analyzer wherein a test sample of the rapidly disintegrating or rapidly dissolving coating is tested for under 25° C./60% relative humidity, 25° C./80% relative humidity and 40° C./75% relative humidity and the slope of the water vapor transmission versus time plot exhibits a slope that is less than 60 under all three test conditions.

6. The pharmaceutical composition as defined in claim 5 wherein the slope of the water vapor transmission versus time plot exhibits a slope that is less than 40 under all three test conditions.

7. The pharmaceutical composition as defined in claim 6 wherein the slope of the water vapor transmission versus time plot exhibits a slope that is less than 20 under all three test.

8. The pharmaceutical composition as defined in claim 1 wherein the core is a core comprising the tolterodine and at least one pharmaceutically acceptable excipient and wherein the core is prepared by extrusion spheronization and does not contain a film forming polymer.

9. The pharmaceutical composition as defined in claim 1 wherein the controlled release coating further comprises a pH dependent material.

10. The pharmaceutical composition as defined in claim 9 wherein the controlled release coating further comprises a first controlled release coating comprising a water insoluble polymer and a pore forming agent and a second controlled release coating comprising a pH dependent polymer.

11. A stable controlled release oral pharmaceutical composition comprising a plurality of particles wherein at least one of the particles consists essentially of:
(a) a core comprising a therapeutically effective amount of tolterodine tartrate and at least one pharmaceutically acceptable excipient;
(b) a rapidly disintegrating or rapidly dissolving coating applied directly to the core that exhibits a low water vapor transmission rate as determined by a water permeability analyzer wherein a test sample of the rapidly disintegrating or rapidly dissolving coating is tested for under 25° C./60% relative humidity, 25° C./80% relative humidity and 40° C./75% relative humidity and the slope of the water vapor transmission versus time plot exhibits a slope that is less than 60 under all three test conditions; and
(c) a controlled release coating applied to the rapidly disintegrating or rapidly dissolving coating wherein the controlled release coating comprises a water insoluble film forming polymer and a pore forming agent.

12. The pharmaceutical composition as defined in claim 11 wherein the slope of the water vapor transmission versus time plot exhibits a slope that is less than 40 under all three test conditions.

13. The pharmaceutical composition as defined in claim 11 wherein the slope of the water vapor transmission versus time plot exhibits a slope that is less than 20 under all three test conditions.

14. The pharmaceutical composition as defined in claim 11 wherein the core is prepared by extrusion spheronization.

15. The pharmaceutical composition as defined in claim 11 wherein the controlled release coating further comprises a pH dependent material.

16. The pharmaceutical composition as defined in claim 11 wherein the controlled release coating further comprises a first controlled release coating comprising a water insoluble polymer and a pore forming agent and a second controlled release coating comprising a pH dependent polymer.

17. A stable controlled release oral pharmaceutical pellet consisting essentially of:
(a) an extruded spheronized core comprising a therapeutically effective amount of tolterodine tartrate, lactose and microcrystalline cellulose;
(b) a rapidly disintegrating or rapidly dissolving coating applied directly to the core that exhibits a low water vapor transmission rate as determined by a water permeability analyzer wherein a test sample of the rapidly disintegrating or rapidly dissolving coating is tested for under 25° C./60% relative humidity, 25° C./80% relative humidity and 40° C./75% relative humidity and the slope of the water vapor transmission versus time plot exhibits a slope that is less than 40 under all three test conditions;
(c) a controlled release coating comprising a water insoluble polymer and a pore forming agent; and
(d) a delayed release coating comprising a pH dependent polymer.

18. The controlled release pellet as defined in claim 17 wherein the rapidly disintegrating or rapidly dissolving coating comprises polyvinyl alcohol.
